(19) United States
(12) Patent Application Publication
(10) Pub. No.: US 2015/0342669 A1
(43) Pub. Date: Dec. 3, 2015

(54) DEVICES AND METHODS FOR CONTROLLED ENERGY DELIVERY TO AIRWAYS

(71) Applicant: Boston Scientific Scimed, Inc., Maple Grove, MN (US)

(72) Inventors: Aiden Flanagan, Kilcolgan (IE); Bryan Allen Clark, Forest Lake, MN (US)

(73) Assignee: Boston Scientific Scimed, Inc.

(21) Appl. No.: 14/723,001

(22) Filed: May 27, 2015

Related U.S. Application Data
(60) Provisional application No. 62/004,621, filed on May 29, 2014.

(51) Int. Cl. A61B 18/14

(52) U.S. Cl. CPC A61B 18/1482; A61B 2018/00023

(57) ABSTRACT

In one embodiment, a device for delivering energy to tissue within a patient may include an elongate member having a proximal end and a distal end, an expandable energy delivery assembly extending from the distal end of the elongate member, wherein the expandable energy delivery assembly includes a plurality of legs, wherein at least one of the legs includes an electrode and defines a passageway adjacent to the electrode, and wherein the passageway is configured to receive a fluid.

### CROSS-REFERENCE TO RELATED APPLICATION

This patent application claims the benefit of priority to U.S. Provisional Application No. 62/004,621, filed May 29, 2014, the entirety of which is incorporated herein by reference.

### FIELD OF THE DISCLOSURE

This disclosure relates generally to methods and devices for treating a patient's lungs to, for example, inhibit or otherwise mitigate symptoms of chronic obstructive pulmonary disorders. More particularly, the disclosure relates to methods and devices for delivering energy to an airway wall of a diseased lung to reduce at least one symptom of chronic obstructive pulmonary disease and asthma.

### BACKGROUND OF THE DISCLOSURE

Chronic obstructive pulmonary disease (COPD) is a progressive disease that affects breathing efficiency and capacity of lungs. COPD includes conditions such as chronic bronchitis and emphysema. COPD currently affects over 15 million people in the United States and is currently a leading cause of death in the country. The primary cause of COPD is inhalation of cigarette smoke, which is responsible for over 90% of COPD cases. The economic and social burden of the disease is substantial and is increasing.

Chronic bronchitis is generally characterized by chronic cough with enhanced sputum production. Due to airway inflammation, mucus hyper-secretion, airway hyper-responsiveness, and/or eventual fibrosis of the airway walls, significant airflow and gas exchange limitations may develop in the lungs. Hence, a patient with COPD may have difficulty in breathing and suffer from shortness of breath.

Emphysema is a long-term lung disease characterized by destruction of the lung tissue (i.e., lung parenchyma). The lung parenchyma is a tissue that supports the shape and function of the lungs. Hence, emphysema leads to loss of elastic recoil and tethering which maintains airway patency, and hence, the ability of the lungs to exhale air is reduced. The destruction of the lung tissue is mainly caused by destruction of alveolar walls. Also, because bronchioles are not supported by cartilage like the larger airways are, they have little intrinsic support and therefore are susceptible to collapse when destruction of tethering occurs, particularly during exhalation.

Further, acute exacerbations of COPD (AECOPD) have significant adverse effects on many COPD patients and are associated with an increase in morbidity and mortality. AECOPD events are defined as acute worsening of symptoms, for example, an increase in or onset of cough, wheeze, and sputum changes that typically last from several days to a couple of weeks. Various factors such as bacterial infection, viral infection, and/or pollutants, trigger AECOPD and may lead to significant airway restriction through one or more of airway inflammation, mucus hypersecretion, and/or bronchoconstriction.

Asthma is a chronic condition characterized by bronchoconstriction, excessive mucus production, and inflammation and swelling of airways. These conditions cause widespread and variable airflow obstructions thereby making it difficult for asthma sufferers to breathe. Asthma may include acute episodes or may be attached to other episodes of additional airway narrowing via contraction of hyper-responsive airway smooth muscle. The chronic nature of asthma can also lead to remodeling of the airway wall (e.g., structural changes such as thickening or edema), which can further affect the function of the airway wall and influence airway hyper-responsiveness. Other physiologic changes associated with asthma include excess mucus production, and if the asthma is relatively severe, mucus plugging, as well as ongoing epithelial denudation and repair. Epithelial denudation exposes the underlying tissue to substances that would not normally come in contact with them, further reinforcing the cycle of cellular damage and inflammatory response.

Despite relatively efficacious drugs e.g., long acting muscarinic antagonists, beta agonists, corticosteroids, and antibiotics that treat COPD and asthma, patients may suffer from frequent exacerbations and may have declined lung function, poorer quality of life, and greater morbidity. Various medical procedures including minimally invasive options are known to reduce excessive mucus secretion and/or bronchoconstriction in patients suffering from chronic bronchitis.

One approach for treatment of COPD and asthma includes controlling parasympathetic nerve signals to the lungs to control bronchoconstriction, mucus secretion, inflammation, and remodeling. In particular, treatment may target parasympathetic neural tissue that controls the airway smooth muscles and mucus secretions. In particular, cholinergic nerve fibers arise in the nucleus ambiguous in the brain stem and travel down the vagus nerve and synapse in parasympathetic ganglia, which are located within and external to the airway wall. These parasympathetic ganglia are most numerous in the trachea and mainstem bronchi, especially near the hilus and points of bifurcations, with fewer and smaller-sized ganglia dispersed in distal airways. From these ganglia, short post-ganglionic fibers travel to airway smooth muscle and sub-mucosal glands. Acetylcholine (Ach), the parasympathetic neurotransmitter, is released from the post-ganglionic fibers and acts on M1- and M3-receptors on smooth muscles and sub-mucosal glands to cause bronchoconstriction and mucus secretion, respectively. ACh is also believed to regulate airway inflammation and airway remodeling, and contribute significantly to obstructive airway diseases such as COPD. Along the same lines, antichlorigenic drugs such as SPIRIVA® are believed to work by competing with ACh for binding sites on receptors on smooth muscle cells, resulting in reduction of bronchoconstriction.

Irritants, such as, e.g., cigarette smoke or pollution, mechanical stimuli and other irritants in the airways can trigger different receptor nerve cells and set-off a reflex action initiating bronchoconstriction and mucus production. The receptor nerve cells include rapidly adapting receptors (RARs) and C-Fibers, both of which have nerve endings in the epithelium.

In addition, nerve cells can also be targeted to treat bronchial hyperreactivity (BHR). BHR is present in a considerable number of COPD patients, constituting as much as 60% to 94% of the patient population. BHR may be caused by hypersensitivity of receptor nerve fibers. As a result of BHR, the thresholds for reflex action initiation and natural self-limitation mechanism of acetylcholine release are reduced. As airway resistance varies inversely with the fourth power of the airway radius, BHR is believed to be a function of both bronchoconstriction and inflammation. Inflammation in the airway walls reduces the inner diameter of the airway lumen, thus amplifying the effect of baseline cholinergic tone.

To break the reflex, a nerve destruction (e.g., ablation by applying heat, transferring energy away from airway tissue, or application of chemicals) therapy or procedure can be performed on a region with high concentration of nerves using techniques such as radiofrequency heating, cryo-therapy, etc. For optimal results with reduced inflammation, ablation should be localized to avoid unnecessary damage to surrounding tissues. In some cases, temperature of the ablation therapy may need to be regulated. Also, the ablation should be localized or otherwise limited both axially as well as radially to avoid unnecessary damage to epithelial and sub-mucosal tissue layers. For this purpose, circulating fluid may be used. However, a circulating cooling fluid may not be able to prevent temperature spikes and avoid damage to surrounding tissues. In some embodiments, the cooling fluid may be replaced with a therapy fluid which itself may be a chemical formulation designed to perform an ablation-like therapy to damage nerves or otherwise disrupt their abilities to transmit signals, as described in greater detail below.

Thus, there remains a need for improved methods and devices that allow for better treatment of COPD and asthma patients during, e.g., an ablation procedure for destroying or otherwise damaging tissues of a lung airway.

### SUMMARY OF THE DISCLOSURE

Embodiments of the present disclosure relate to devices and methods of treating airways.

In one embodiment, a device for delivering energy to tissue within a patient may include an elongate member having a proximal end and a distal end, an expandable energy delivery assembly extending from the distal end of the elongate member, wherein the expandable energy delivery assembly includes a plurality of legs, wherein at least one of the legs includes an electrode and defines a passageway adjacent to the electrode, and wherein the passageway is configured to receive a fluid.

Various embodiments of the device may include one or more of the following features: at least one sensor configured to detect temperature and/or impedance; distal ends of the plurality of legs are coupled together to define a basket or a stent; each of the plurality of legs includes a free distal end; at least one of the legs includes a plurality of passageways adjacent the electrode; a wall of the passageway includes an opening through which the fluid can leave the passageway; the opening is configured to cause a combination of gas expansion cooling and/or evaporative cooling; the opening is configured to cause the fluid to undergo an adiabatic phase change; the passageway includes a first cross-sectional configuration and the electrode includes a second cross-sectional configuration different from the first cross-sectional configuration; the opening includes a plurality of openings disposed along a wall of the passageway; the temperature sensor is disposed on the at least one of the legs; and the fluid is configured to receive thermal energy from tissue within the patient, wherein the expandable energy delivery assembly is bipolar; the a controller configured to adjust the delivery of energy based on information received from the at least one sensor; the fluid comprises nitrous oxide and/or nitrogen; and the energy if RF energy.

In another embodiment, a device for delivering energy to tissue within a patient may include an elongate member including a proximal end and a distal end, a plurality of legs defining an energy delivery assembly, wherein the energy delivery assembly is configured to selectively transition between an expanded configuration and a collapsed configuration, wherein at least one of the plurality of the legs includes an electrode and a passageway for receiving a cooling fluid therein adjacent the electrode, and a sensor configured to detect temperature and/or impedance.

Various embodiments of the device may include one or more of the following features: the passageway includes two passageways disposed on either side of the electrode; each of the plurality of legs includes a distal end; the distal ends of the plurality of legs are coupled together to form an expandable basket; the distal ends of the plurality of legs are not coupled together; a wall of the passageway includes an opening through which the fluid can leave the passageway, wherein the opening is configured to cause the fluid to undergo an adiabatic phase change; and the energy delivery assembly is bipolar.

In another embodiment, a method of treating an airway of a patient may include delivering energy to a wall of the airway via a device. The device may include an elongate member including a proximal end and a distal end, a plurality of legs defining an energy delivery assembly, wherein the energy delivery assembly is configured to selectively transition between an expanded configuration and a collapsed configuration, wherein at least one of the plurality of the legs includes an electrode and a passageway for receiving a cooling fluid therein adjacent the electrode, and a sensor configured to detect temperature and/or impedance.

Various embodiments of the method may include one or more of the following features: the energy delivery assembly may be bipolar, and wherein the opening is disposed on at least one of the plurality of electrodes; and a wall of the passageway includes an opening through which the fluid can leave the passageway, wherein the opening is configured to cause the fluid to undergo an adiabatic phase change.

Additional characteristics, features, and advantages of the disclosed subject matter will be set forth in part in the description that follows, and in part will be apparent from the description, or may be learned by practicing the disclosure. The characteristics and features of the disclosure can be realized and attained by way of the elements and combinations particularly pointed out in the appended claims.

It is to be understood that both the foregoing general description and the following detailed description are exemplary and explanatory only, and are not restrictive of the disclosed subject matter.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 is a schematic view of a device deployed in an airway for delivering energy to a patient's tissue, according to one embodiment of the present disclosure;

FIG. 2A is a schematic view of an exemplary energy delivery device deployed in an airway, according to another embodiment of the present disclosure;

FIG. 2B is a schematic view of an exemplary energy delivery device deployed in an airway, according to another embodiment of the present disclosure;

FIG. 3A shows a schematic view of an exemplary basket leg, according to an embodiment of the present disclosure;

FIG. 3B shows a schematic view of an exemplary electrode with a passageway for a fluid, according to an embodiment of the present disclosure;

FIG. 4 is a schematic view of another exemplary basket leg, according to another embodiment of the present disclosure; and

FIG. 5A is a schematic view of a device deployed within an airway of a patient's lung, according to an embodiment of the present disclosure.

FIG. 5B is a schematic view of an exemplary energy delivery device deployed in an airway, according to another embodiment of the present disclosure;

FIG. 5C is a schematic view of an exemplary energy delivery device deployed in an airway, according to another embodiment of the present disclosure;

FIG. 6A is a schematic view of a device configured to be deployed within an airway of a patient's lung, according to another embodiment of the present disclosure.

FIG. 6B is a schematic view of a device configured to be deployed within an airway of a patient's lung, according to another embodiment of the present disclosure.

### DESCRIPTION OF THE EMBODIMENTS

Reference will now be made to certain exemplary embodiments of the present disclosure, examples of which are illustrated in the accompanying drawings. Wherever possible, the same reference numbers will be used throughout the drawings to refer to the same or like parts. The term “distal” refers to the end farthest away from a medical professional when introducing a device in a patient. The term “proximal” refers to the end closest to the medical professional when placing a device in the patient.

The term “diathermy therapy” includes a technique of generating heat by delivering energy and using the generated heat for medical purposes. Diathermy can be achieved using ultrasonic energy and/or different types of electromagnetic energy such as microwave energy, radio-frequency, or the like.

### Overview

Embodiments of the present disclosure relate to devices and methods configured for transferring or otherwise delivering thermal energy to walls of body lumens and/or human tissue. Each of the disclosed embodiments may include one or more of the features disclosed in connection with any of the other disclosed embodiments without departing from the principles contemplated herein.

One aspect of the disclosed devices and/or methods may be used for delivering thermal energy to an airway wall in a lung in a controlled manner to, among other things, reduce the number of parasympathetic nerves in the layers of tissue within or on the surface of the airway wall, reduce airway smooth muscle, reduce mucus gland activity, ablate cancer lesions, or otherwise reduce/eliminate unwanted airway tissue. Using the disclosed devices and methods, the parasympathetic nerves may be targeted for denervation (reduction in the activity or death) or interference with the reflex pathway. That is, the nerves in the airway may be destroyed, eliminated, or otherwise damaged so as to be rendered wholly or partially inoperable for their native functions. The parasympathetic nerves in both upper airways (trachea, main stem bronchioles) and distal branches of the parasympathetic nerves can be targeted. By reducing the number and activity of nerve receptors, and/or nerve cells, at least one symptom of COPD, including, but not limited to, bronchoconstriction and/or mucus secretion may be abated. The temperature of the airway wall may increase due to application of energy during a diathermy or other procedure. The temperature rise may be restricted to the outer layers of the airway wall by controlling a rate of energy delivered to the tissues of the airway. Energy delivered to the airway may be accomplished through the use of selective cooling via, e.g., a cooling fluid as discussed in greater detail below. If the temperature of the airway wall may be controlled using a cooling fluid before, during or after diathermy procedure, the damage to surrounding tissue (e.g., the inner layers of the airway wall) may be reduced or otherwise limited. As described below also, the cooling fluid may be replaced by a therapeutic fluid capable of performing, e.g., a chemical ablative procedure to damage or otherwise disrupt nerve tissue within the airways.

### Exemplary Embodiments

The embodiments disclosed herein include methods and devices for treating a respiratory airway. However, it should be noted that the present disclosure contemplates use of the methods and devices for treatment of other body regions and/or tissue, such as renal nerves, bladder tissue, or the like. In addition to diagnosed airway diseases e.g., COPD, asthma, chronic cough, chronic bronchitis, and cystic fibrosis, other diseases such as bronchial hyperactivity associated with congestive heart failure and mitral valve stenosis also can be treated using the devices and the methods disclosed herein.

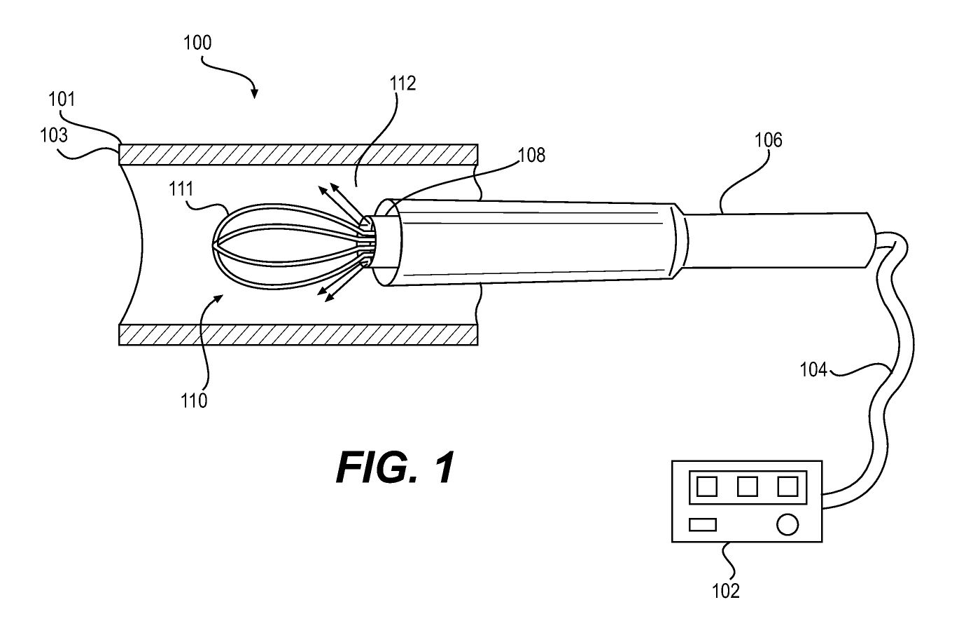

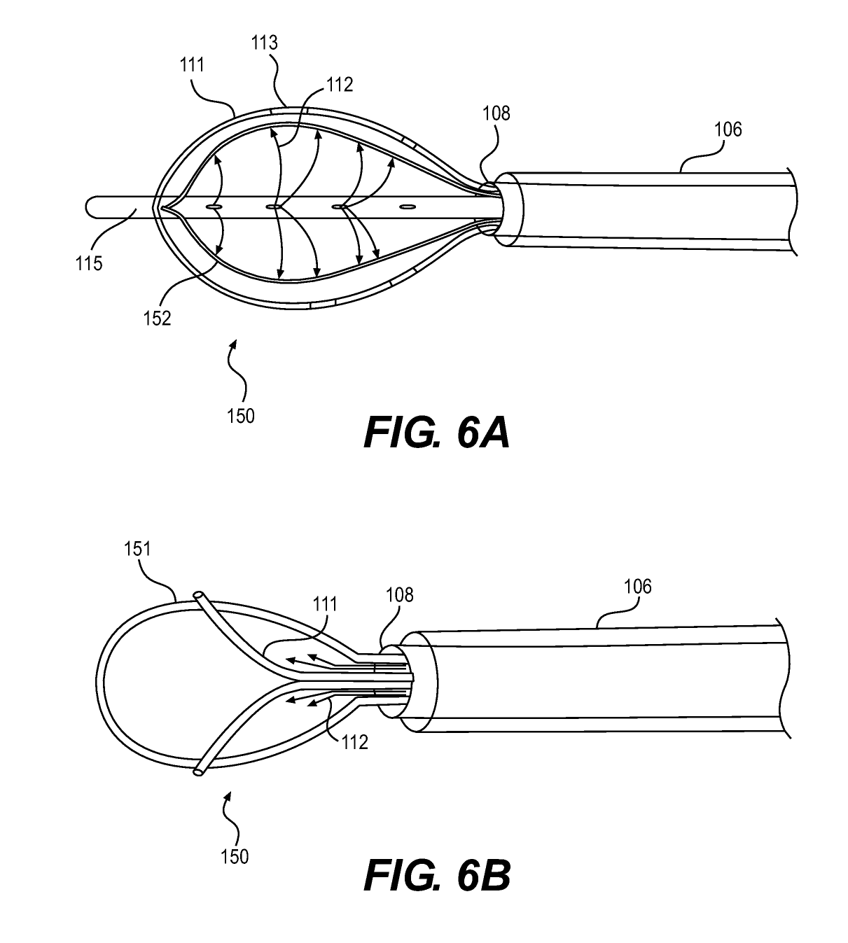

FIG. 1 is a schematic view of an exemplary device 100 deployed in a body lumen, such as, e.g., an airway 101 of a patient's lung. The device 100 may be configured to deliver thermal energy to, among other things, denervate the airway wall 103 and deliver a cooling fluid 112 to the airway 101 that may help regulate the temperature of the airway wall 103 during the therapy or the procedure. As discussed above, the fluid 112 may be replaced with a therapeutic fluid that is chemically active and capable of performing a chemical ablative procedure for destroying or otherwise damaging nerve tissue in airway walls. Structurally, the device 100 may include an elongate member 108 and an expandable energy delivery assembly such as an expandable basket 110. In some embodiments, the expandable basket 110 may be replaced with one or more inflatable balloons 150 (FIGS. 6 and 8) having electrodes disposed on an outer surface of the balloon 150. In some embodiments, the expandable basket 110 may be replaced with an expandable stent or spiral or lasso-shaped catheter (not shown) having one or more electrodes disposed on an outer surface of the expandable stent or catheter. In one embodiment, a spiral catheter may be configured to transition from a elongate configuration to a spiral and/or coiled configuration.

As shown in FIG. 1, the elongate member 108 may be a generally flexible, hollow, tubular-member having a circular cross-section with a proximal end and a distal end. Alternatively, other cross-sectional shapes including cylindrical, rectangular, oval, or other suitable shapes also may be contemplated as appropriate for use in the intended environment. The elongate member 108 may define a single lumen as shown in FIG. 1, or a plurality of lumens, as desired. The lumen(s) within the elongate member 108 may extend through the entire length of the elongate member 108 or only extend partially through the elongate member 108. One or more of the lumens within elongate member 108 may be operably coupled to a port, which may facilitate the introduction of surgical instruments into the lumens of elongate member 108.

The elongate member 108 may be formed of a suitable biocompatible material such as, but not limited to, polymers, metals, alloys, or other suitable flexible materials known to those skilled in the art. Exemplary materials include nylon, silicone, Polytetrafloroethylene (PTFE) or Teflon™, Polyethylene (PET), Low Density Polyethylene (LDPE), or the like. Such materials may be used either alone or in a combination to form the elongate member 108. In some embodiments, an outer surface of the elongate member 108 may include a lubricious coating that may allow smooth passage of the elongate member 108 through an introduction member 106 such as an endoscope, a bronchoscope, or a catheter. Additionally, in some embodiments, the elongate member 108 may have an antibacterial coating on its outer surface. Further, in some embodiments, a distal end portion of elongate member 108 may be steerable or selectively positionable. In such embodiments, elongate member 108 may include an appropriate steering mechanism, such as, e.g., pull wires operably coupled to a proximal actuator. In other embodiments, elongate member 108 may include a plurality of articulating links coupled to one another to facilitate selective positioning of elongate member 108 within a patient's body.

The expandable basket 110 may be coupled to the distal end of the elongate member 108. In another embodiment, the expandable basket 110 may be configured to extend out of and withdraw into a lumen of elongate member 108, as shown in FIG. 1. In the illustrated embodiment, e.g., the expandable basket 110 may be configured to transition between an expanded configuration and a collapsed configuration. To this end, the device 100 may include an actuation mechanism (not shown) that may facilitate the transition of the expandable basket 110 between the expandable and the collapsed configurations. An example of such actuation mechanisms may include a push-pull member such as a wire (or other suitable control element) that may further be coupled to a handle at its proximal end and the expandable basket 110 at a distal end. The handle may be manipulated to control the push and pull motion of the push-pull member, thereby controlling the expandable basket 110 to expand and collapse, respectively. As will be discussed below in greater detail, the expandable basket 100 may include a plurality of legs 111. The legs 111 may be configured to self-expand when extended out of elongate member 108, thereby forming the “basket” shape of expandable basket 100.

In some embodiments, legs 111 (which may form electrodes of the expandable basket) may be coupled together at their proximal ends to the above-mentioned push-pull member. The distal ends of the legs 111 also may be coupled to one another, as shown in FIG. 1. The distal end of the expandable basket 110 may have a generally rounded-configuration or a blunt configuration that may remain atraumatic while the device 100 may be introduced through the airway 101. The legs 111 may have one or more active electrode regions on its surface. In some embodiments, a portion of one or more legs 111 may include an insulating covering (not shown) adjacent an active region, as discussed below in greater detail.

In the expanded configuration, the legs 111 may be configured to expand radially to form a basket like structure. In contrast, the legs 111 may remain parallel to the longitudinal length of the elongate member 108 in the collapsed configuration such that once collapsed, the expandable basket 110 can retract into the elongate member 108. In some embodiments, where contact or close proximity with the airway wall 103 is beneficial, the radial expansion of the expandable basket 110 may allow some arcuate sections of the legs 111 of the expandable basket 110 to come in contact with an inner surface of the airway wall 103. Those skilled in the art will appreciate that the expandable basket 110 may take on any suitable shape such as, but not limited to, oblong, spherical, cylindrical, or the like.

In some embodiments, the legs 111 may be made of an electrically conductive material, such as, but not limited to, Nitinol™, stainless steel or the like. In a first instance, one or more of the plurality of legs 111 may be coated with an insulating material, with discrete areas of insulation later removed (or merely omitted) to form electrically active electrode regions. In a second instance, the legs 111 may be coated with an insulating material and individual electrodes may be affixed to the legs 111. In addition, individual electrodes also may be affixed directly to the legs 111. In general, the electrically active regions may be positioned to contact the tissue of the inner surface of the airway wall 103 to deliver thermal energy. If using microwave energy for diathermy, then the electrically active regions can be centrally located within the airway 101 and may not need to contact the airway wall 103. Using microwave energy for diathermy, relatively fast temperature rise and shorter procedure times may be possible.

In the illustrated embodiment, the expandable basket 110 may have four legs 111, however, it should be noted that any suitable number of legs 111 such as, one, two, three, and so forth may be contemplated. In some embodiments, the legs 111 of the expandable basket 110 may be spaced apart uniformly; however, other embodiments may include the legs 111 spaced apart at a non-uniform distance.

Because the target nerves may be within the airway wall 103 or on the inner surface of the airway wall 103, diathermy involves a high risk of damage to surrounding tissue (e.g., an inner tissue layer of the airway wall). Without any cooling mechanism or otherwise controlled energy delivery, the thermal energy provided through the electrodes may cause the electrode to overheat the airway wall 103. In at least some examples, the energy delivered (e.g., RF energy) from an electrode or other energy delivery element, may heat tissue by passing alternating current through the tissue. This may cause ion movement in the tissue, which may result in molecular frictional heating of the tissue. The heated tissue also may heat the electrode. To prevent such overheating, the present disclosure employs a cooling fluid 112 that may regulate the temperature of the airway wall 103 and the electrode, thereby enabling relatively fast heating of tissue in a desired location without spread of thermal damage to inner tissue layers of the airway wall 103. The cooling fluid 112 may be applied near the diathermy treatment location through one or more cooling fluid channels defined along a length of the elongate member 108. The cooling fluid channel(s) may include one or more cooling fluid channels and/or openings (not shown) through which the disposed cooling fluid 112 may be injected or sprayed proximate the treatment location. By applying the cooling fluid 112 by injection and/or spraying the cooling fluid 112, there is no need for a return cooling fluid channel, such as used with circulating coolants e.g., saline. In addition, the cooling fluid 112 may be provided as a coating on an external surface of a balloon 151 (FIGS. 6A and 6B). The balloon 152 may be inserted into a patient's airways and expanded so as to place the external surface into contact with a wall of the airway, thereby delivering the cooling fluid 112 to the tissues of the airway walls. As discussed herein, the cooling fluid 112, may be replaced with another fluid configured to perform chemical ablation of airway tissue. For example, an adiabatic change may occur inside of an air or vapour filled balloon having a balanced exit lumen to keep pressure in the balloon constant. The orifices injecting the fluid into the balloon may be disposed on a central lumen within the balloon, or on the circumferential ends of the balloon. This may cool the balloon surface to in turn cool the airway wall that the balloon surface is in contact with. Adiabatic change also may occur within an electrode itself at a point where the electrode contacts tissue.

In some embodiments, the cooling fluid 112 may exit the elongate member 108 through one or more narrow openings, orifices or valves (not shown). Because of the Joule-Thomson effect, the cooling fluid 112 may cool as it expands after passing through one or more openings. In some embodiments, the openings may be configured to atomize the cooling fluid 112, thereby enhancing the evaporative cooling effects. In some embodiments, the cooling fluid 112 may undergo an adiabatic phase change as the cooling fluid 112 expands through the opening, changing from a liquid phase to a gas phase. The cooling fluid 112 may undergo a phase change due to a sudden drop in pressure, which may drop the temperature of the cooling fluid 112 significantly. As such, the cooling fluid 112 may be configured to absorb heat from tissues of the airway wall 103.

The cooling fluid 112 may be chosen such that the cooling fluid 112 may be biocompatible and may have a low boiling point. Exemplary embodiments include nitrous oxide, nitrogen, etc. Once the cooling fluid 112 comes in contact with the airway wall 103, the cooling fluid 112 may lower temperature of the airway wall 103 by absorbing heat energy from the airway wall 103, which may avoid overheating of the airway wall 103, thereby preventing any undesired tissue damage. It may be noted that the cooling fluid 112 may reduce the temperature of the airway wall 103 to maintain the temperature of the airway wall within a range of 0° C. to 45° C. In this manner, high capacity cooling can be applied so that a high energy can be input by the basket while maintaining cool airway tissue, thereby creating a faster therapy, less heat conduction and more localized heating. In some embodiments, the cooling fluid 112 may further reduce the temperature of the airway wall within a range of −5° C. to −15° C., where at most a limited apoptosis occurs in a small fraction of cells. The cooling caused as a result of the application of the cooling fluid (e.g. via spray, injection, etc.) may be effective enough to cool deeper layers of the airway wall 103. In another example, cooling fluid 112 may be a low boiling point fluid that can be sprayed on the airway wall. In yet another example, the evaporative cooling fluid may have a higher boiling point which may be applied as a fine mist to the airway wall.

To control the temperature and flow of the cooling fluid 112, the elongate member 108 may be connected to a console unit 102 outside patient's body through one or more connecting conduits or cables 104. The console unit 102 may include a controller, a power unit, and an interface for monitoring parameters such as voltage, current, pulse, temperature, impedance, pressure or the like. In some embodiments, expandable basket 110 may include one or more sensors configured to measure one or more of voltage, current, pulse, temperature, impedance, and/or pressure. In some embodiments, the console unit 102 may control a RF electrode or a microwave electrode for diathermy therapy. Electrode control may be based on feedback provided by the aforementioned sensor(s) provided on expandable basket 110. An example of feedback energy control is described below in greater detail. It may be further contemplated that diathermy can be achieved using resistance heating, ultrasonic, laser devices, etc., each of which may be controlled in a similar manner. The console unit 102 may be electrically coupled to the expandable basket 110 through one or more electrically conducting wires or conductors. The conductors may be routed through one of the lumens of the elongate member 108 and may be connected to the expandable basket 110. In some embodiments, the conductors may form a unitary structure with the legs 111 of the expandable basket 110 e.g., monolithically formed extending proximally from the legs 111. At a proximal end of the conductor, one or more connectors may be disposed to support a connection between the console unit 102 and the device 100.

The device 100 may be used for delivering energy in bipolar or monopolar mode. For monopolar mode, one or more electrodes may be supplied with single polarity of energy by connecting the electrodes to a single pole and an external electrode e.g., a pad electrode, may be connected to an opposite pole. Thus, in some embodiments, the legs 111 containing the active electrode regions of the expandable basket 110 can function as one pole and an external electrode (placed, e.g., under the patient) will be another pole and connected to another part of the patient's body to complete the circuit. On the other hand, for bipolar mode of energy delivery, multiple electrodes may be attached to the opposite poles of energy source. In other embodiments, active electrode regions on the same or different legs 111 of the expandable basket 110 can function as opposite poles and there is no need for an external electrode. Energy in bipolar mode may be targeted between the two poles and hence, desired effect of the therapy may be achieved with less energy and more control of the location that is heated.

In some embodiments, the console unit 102 may contain a temperature feedback mechanism to regulate the temperature in the airway 101. Such a feedback mechanism may help avoid excessive heating or cooling of the airway wall 103. A feedback processor may be deployed to receive information from the temperature sensors and process the signal accordingly. Based on the processed feedback, the console unit 102 may allow release of the cooling fluid 112 or increase/decrease energy delivered to the electrode in order to regulate the therapy. The temperature sensors may be disposed at various locations of the device 100, including on the electrodes or the legs 111 of the expandable basket 110, near the active electrode region, or on the outer surface or the lumen of the elongate member 108, etc. In some embodiments, only one temperature sensor may be deployed to detect the temperature, however, in other embodiments, each leg 111 of the expandable basket 110 may have a temperature sensor. In some embodiments, the temperature sensor may be placed in contact with the airway wall 103 to detect the temperature of the airway wall 103 tissue.

The device 100 may also include temperature sensors such as, but not limited to, thermistors, thermocouples, IR sensors, and/or resistance temperature detectors, to detect one or more temperatures of tissues within the airway wall 103. The thermocouples used as temperature sensors may be formed using a combination of materials such as, but not limited to, Nickel-Aluminium, Nickel-Chromium, Constantan-Iron, Constantan-Copper, or the like.

In some embodiments, the introduction member 106 such as, but not limited to, a delivery sheath, catheter, or a bronchoscope may be employed to introduce and advance the device 100 to a desired treatment location. The device 100 may be dimensioned so as to pass through a working channel of the introduction member 106. However, in some embodiments, the device 100 may be directly advanced into the airway 101.

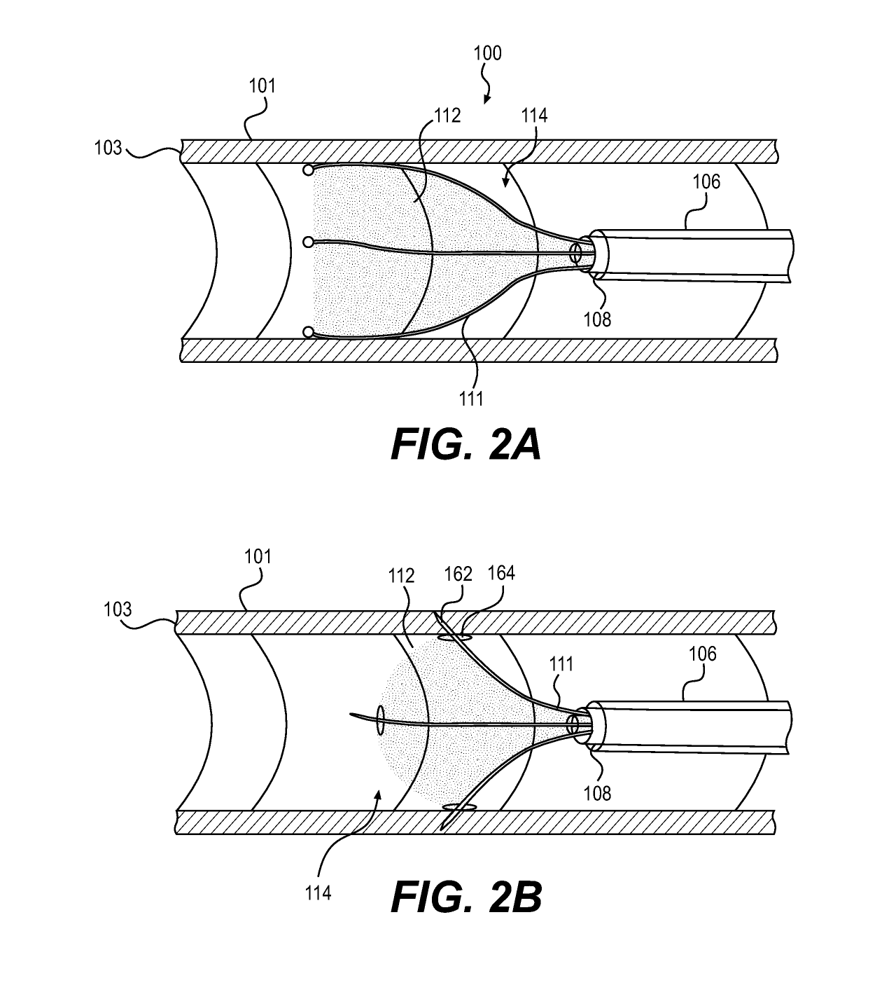

FIG. 2A is a schematic view of another embodiment of the device 100 deployed in the airway 101 of a patient's lung. As discussed above, the device 100 may include an expandable energy delivery assembly 114, the elongate member 108, and the introduction member 106 (e.g., a catheter or other suitable introduction sheath).

A plurality of legs 111 may define a plurality of the electrodes that may form the expandable energy delivery assembly 114. Although FIG. 2A depicts that each of the plurality of legs 111 terminate in a distal free end, those of ordinary skill in the art will recognize that one or more of the plurality of legs 111 may be extended and coupled to one another so as to define a distal joint.

In some embodiments one or a portion of the plurality of legs 111 may be configured to come into contact with, come into contact with and distend, and/or come into contact with and penetrate the inner surface of the airway wall. Accordingly, some of the plurality of legs 111 may include a sharper distal end to penetrate the surface. Some of the legs 111 may also include a ‘stop’ mechanism for controlled depth of penetration. In other embodiments, the plurality of legs 111 may have any suitable shape, such as a stent shape for improved control and attachment to the airway tissue.

Expandable energy delivery assembly 114 may include a basket configuration similar to that of expandable basket 110 shown in FIG. 1. That is, the distal ends of legs 111 may be connected to one another. In some embodiments, however, the principles of the present disclosure contemplate that expandable energy assembly 114 includes legs 111 having distal ends that are not connected to one another, as shown in FIG. 2A, to form a plurality of radially-spaced apart prongs. As shown in FIG. 2A, the legs 111 may be disposed relatively close to each other as they extend out of the distal end of elongate member 108 and then gradually move radially away from one another toward the airway wall 103. In some embodiments, the legs 111 may be coupled to each other at a proximal end of the expandable energy delivery assembly 114 and may extend in a distal direction substantially parallel to each other. Such a profile may form a substantially circular cross-section configuration that may conform to the shape of airway such as the airway 101. Those of ordinary skill in the art will understand that assembly 114 may include a profile of any suitable cross-sectional shape, including “D” shaped and/or oval shaped such that assembly 114 may be configured to conform to a plurality of airways having varying cross-sectional configurations. The expandable energy delivery assembly 114 may be configured to transition between an expanded configuration and a collapsed configuration. Further, in the expanded configuration, the legs 111 may come in contact with the airway wall 103 to deliver thermal energy. In the illustrated embodiment, the expandable energy delivery assembly 114 may have a prong-shaped structure while in a partially or fully expanded configuration. However, a person skilled in the art may contemplate any other suitable shape or structure for the expandable energy delivery assembly 114.

In some embodiments, the cooling fluid 112 may be injected into the airway 101 through (e.g., a lumen of) the elongate member 108 that may help in regulating the temperature of the airway wall 103 before, during and/or after diathermy therapy. In some embodiments, the cooling fluid 112 may undergo a liquid to gas phase change and may be injected in the airway 103. In some embodiments, the electrodes (or the legs 111) may define one or more cooling fluid channels through which the cooling fluid 112 may be applied (e.g. injected, sprayed, etc.) into the airway 101, as will be discussed in greater detail below.

In some embodiments, the cooling fluid channel may be located within the legs 111 or the electrodes. In some embodiments, the legs 111 may include one or more openings configured to spray the cooling fluid 112, where the opening may be located adjacent the active electrode region. The opening(s) may be configured to act as a nozzle to facilitate the Joule-Thomson phase change described earlier.

In some embodiments, the expandable energy delivery assembly 114 may include the temperature sensors on the legs 111, near the active electrode region. As discussed above, the temperature sensors may include temperature sensing elements such as thermocouples and/or thermistors, however, other suitable temperature sensors such as IR sensors, or the like may be also contemplated. As alluded to above, the temperature sensors may be disposed at any suitable location on device 100.

FIG. 2B shows an assembly 114 similar in most respects to the energy delivery assembly 114 in FIG. 2A. In the assembly 114, a distal tip 162 of one or more of the legs 111 may be configured to penetrate tissue of the airway wall 103 at any suitable depth. One or more the legs 111 also may include a stop 164 proximal to the distal tip 162 and configured to prevent further penetration of the distal tip 162 into tissue of the airway wall 103. The stop 164 may have any suitable shape, size, and geometry and may be manufactured using any suitable materials.

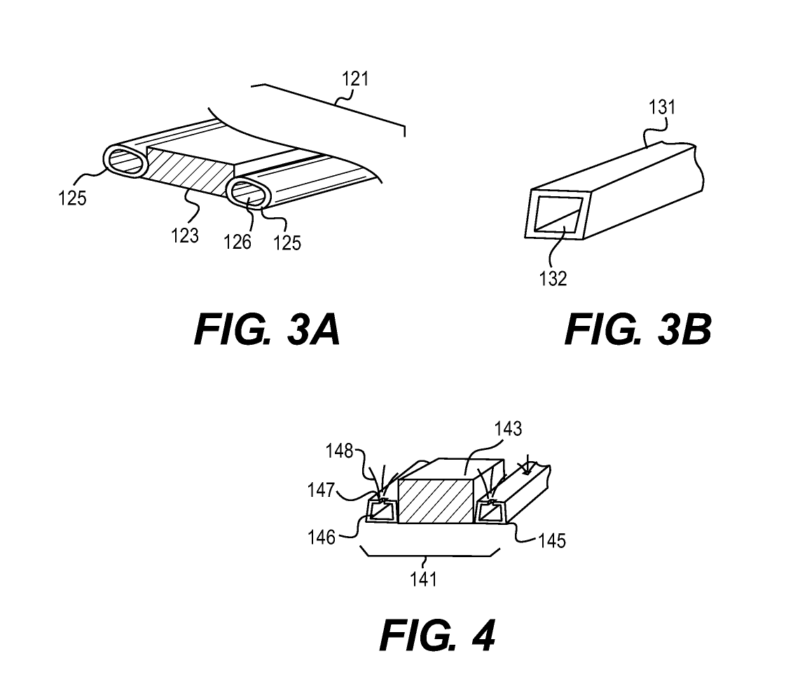

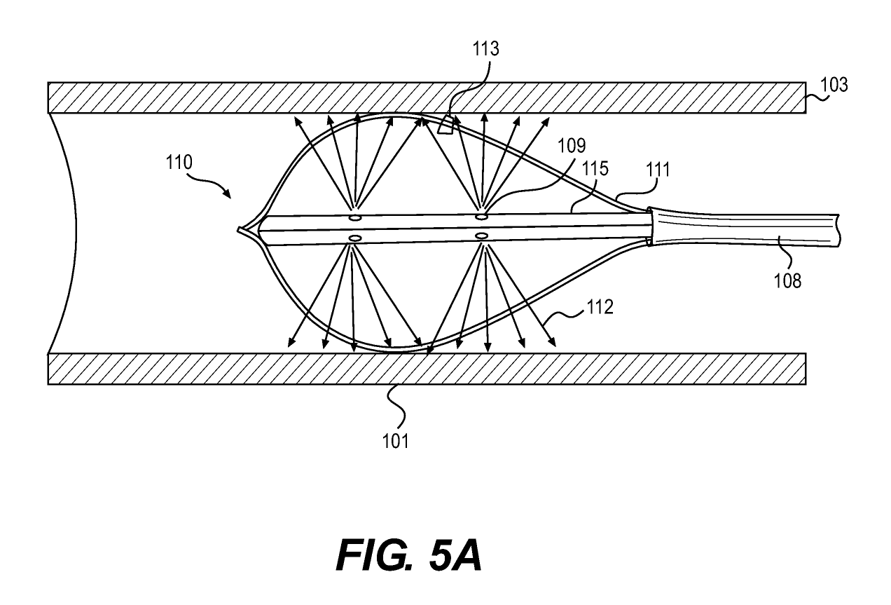

FIGS. 3A, 3B, and 4 show schematic views of various embodiments of electrodes and cooling fluid channels or passageways, in accordance with aspects of the present disclosure.

Referring to FIG. 3A, an exemplary basket leg 121, e.g., may include an electrode 123 with a pair of tubular members 125 that may define one or more passageways 126 for cooling on both sides of the electrode 123. In some embodiments, the tubular members 125 may be disposed adjacent the electrode 123. Although the depicted embodiment illustrates a pair of tubular members 125, those of ordinary skill in the art will understand that a greater or lesser number of tubular members 125 may be used in conjunction with electrode 123. The passageways 126 may be configured to receive the cooling fluid from outside the patient's body and deliver it to the treatment location. Cooling of the airway may occur due to conduction through the walls of the tubular member 125 and/or by injection or spraying of the cooling fluid. The tubular member 125 may conduct cooling to the airway wall. That is, heat energy may travel through the walls of tubular member 125 into the cooling fluid disposed therein. In some embodiments, the tubular member may include an opening adjacent the electrode 123 to release the cooling fluid into the airway. Additionally, in some embodiments, the tubular member 125 may include one or more openings formed thereon. The opening(s) may spray cooling fluid adjacent the electrode 123.

In some embodiments, the tubular members 125 may have an oblong cross-section. In other embodiments, the tubular members 125 may have other cross-sectional shapes such as, e.g., circular, rectangular, square, elliptical, oval, or the like. The tubular members 125 may be formed of electrically insulative material to isolate them from the adjacent electrode 123. The tubular members 125 may be formed of one or more electrically insulative biocompatible polymer such as, but not limited to, Teflon™, silicone, polyurethane, LDPE, HDPE, polycarbonate, or the like. However, the tubular members 125 may be thermally conducting to cool the adjacent electrode 123. Although the depicted embodiment illustrates the electrode 123 disposed in between tubular members 125, one or more tubular members 125 may be disposed between adjacent electrodes 123 (not shown).

As shown in FIG. 3A, the electrode may 123 have a low profile and may be located between a plurality of tubular members 125. Such electrodes may be a microwave electrode centrally located within the airway and may deliver microwave energy radially along a circumference of the airway wall.

In FIG. 3B, the electrode 131 may be hollow and may define a central passageway 132 for receiving and delivering the cooling fluid. Although a single, central passageway 132 is depicted, any suitable number of passageways may be provided within electrode 131. The passageway 132 and the cooling fluid may be electrically isolated from the electrode 131 by incorporating an insulative layer within the body of the electrode 131. That is, the wall of passageway 132 may include an insulating material. By combining the electrode 131 and the passageway 132 into a unitary structure, manufacturing of the device may be simplified. Also, the overall profile of the energy delivery device may be reduced. The electrode 131 may be similar in form and function as that of the electrode 123 shown in FIG. 3A. In some embodiments, an opening may be defined adjacent to a distal end of the electrode 131 to deliver the cooling fluid within airway 101. Those of ordinary skill, however, will understand that one or more openings may be disposed along a length of electrode 131 to spray or otherwise deliver cooling fluid to airway 101.

In FIG. 4, another electrode 143 with hollow members 145 is shown. The exemplary leg 141 may include an electrode 143 with a larger profile (e.g., height, width, cross-sectional dimension, and/or overall configuration) than that of the electrode 131 shown in FIG. 3A. The electrode 143 may be an RF electrode or a microwave electrode. In scenarios where the electrode 143 is an RF electrode, e.g., the electrode 143 may come in contact with the airway wall 103 to deliver thermal energy.

The hollow members 145 may be disposed adjacent the electrode 143. As shown, the hollow members 145 may have a trapezoidal cross-sectional configuration. However, hollow members 145 may have any suitable cross-sectional configuration. Along with a passageway 146 to receive a cooling fluid, the hollow members 145 may also include one or more orifices 147 defined along a longitudinal length of the hollow member 145. The orifices 147 may be configured to deliver the cooling fluid spray 148 adjacent the electrode 143. The size, shape and pattern of the orifices 147 may be defined such that the cooling fluid may undergo an adiabatic phase change such as liquid to gas, as described above.

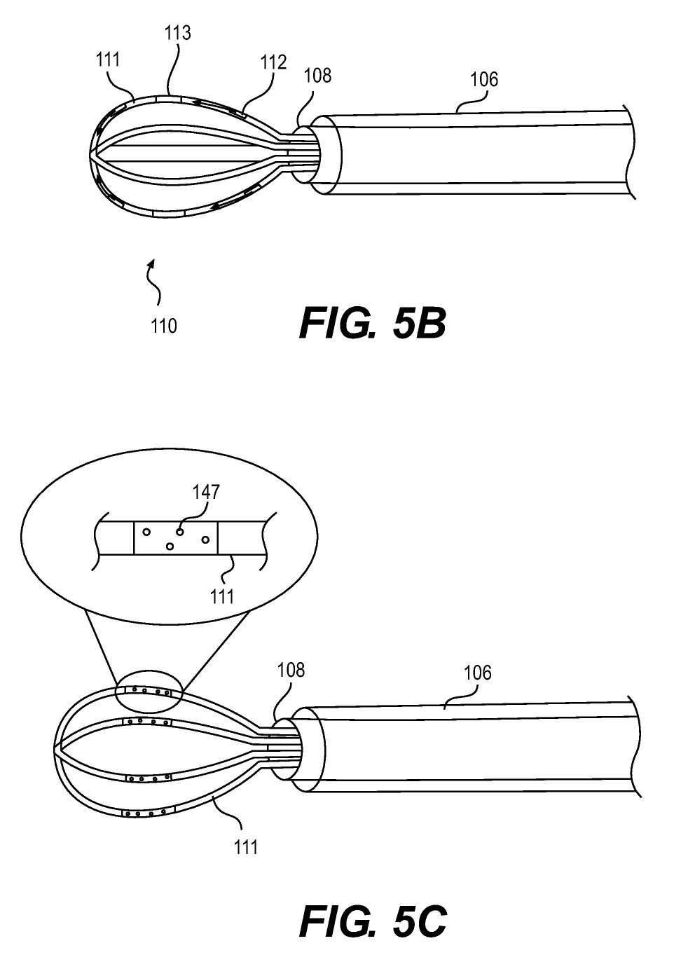

FIG.5A is a schematic view of another embodiment of the device 100 deployed in the airway 101 of a patient's lung. The device 100 may include the expandable energy delivery assembly 110 and the elongate member 108. As described above, the expandable energy delivery assembly 110 may be configured to extend from and withdrawn into a lumen of the elongate member 108. In some embodiments, the expandable energy delivery assembly 110 may include a plurality of electrodes defined by the legs 111. The legs 111 or the electrodes defined by the legs 111 may include active electrode regions for performing diathermy therapy. Thus, the electrodes may be configured to deliver energy to the airway wall 103 during the diathermy therapy. In the illustrated embodiment, the expandable energy delivery assembly 110 may include two legs 111; however, any other suitable number of legs such as three, four, six, eight, or the like also may be contemplated. The legs 111 may also include a thermocouple or other suitable temperature sensors 113 thereon for sensing temperature of the airway wall 103 during the therapy.

One of the lumens of the elongate member 108 may include an actuation mechanism, for example, a pull rod 115 connected to the distal tip of the expandable energy delivery assembly 110. A user may use the pull rod 115 to transition the expandable energy delivery assembly 110 between an expanded configuration and a collapsed configuration. In some embodiments, the pull rod 115 may be formed of conducting material configured to deliver electrical energy to the electrodes to activate the electrodes. The pull rod 115 may define one or more lumens therein. At least one of the lumens of the pull rod 115 may be configured to deliver cooling fluid to the airway, as discussed in greater detail below.

In some embodiments, the pull rod 115 and the expandable energy delivery assembly 110 may extend together to a distal tip. The distal end of the pull rod 115 may be coupled to the expandable energy delivery assembly 110. The pull rod 115 may include one or more lumens that may extend along a longitudinal length of the pull rod 115. The lumens may define plurality of cooling fluid channels configured to pass a cooling fluid 112. The pull rod 115 may also include one or more openings 109 to spray the cooling fluid 112 radially out from the pull rod 115 toward the airway wall 103 or towards the inner diameter of a balloon which may be in contact with the airway wall.

In some embodiments, a user may push and/or pull the elongate member 108 (via, for example, a handle outside of the patient's body) to cause linear movement of the device 100 in the airway 101 towards the treatment location. This mechanism may allow a user to manipulate the elongate member 108 within the airway 101 to access multiple treatment locations.

FIG. 5B is a schematic view of another embodiment of a device which may be deployed in an airway of a patient's lung. The expandable energy delivery assembly 110 of the device may include an the elongate member 108, and a basket having a plurality of electrodes defined by legs 111. The legs 111 as shown in FIG. 5B are similar to the legs 111 shown in FIG. 5A and described above. One or more of the legs 111 shown in FIG. 5B may include a lumen (not shown) through which the cooling fluid 112 may flow so as to cool portions of the assembly 110 of FIG. 5B.

FIG. 5C shows an assembly 110 similar in most respects to the assembly in FIG. 5A, except the legs 111 of FIG. 5C or the electrodes defined by the legs 111 of FIG. 5C may include one or more lumens through which the cooling fluid may flow and openings 147 through which the cooling fluid 112 may be delivered.

FIG. 6A is a schematic view of another embodiment which may be deployed in the airway 101 of a patient's lung. The expandable energy delivery assembly 150 of the device 100 of FIG. 6A may include an expandable balloon 152 and the elongate member 108. The balloon 152 of may be configured to receive cooling fluid within an internal portion of the balloon 152, for example, from the lumen of the elongate member 108. The cooled balloon 152 may be configured to contact the airway wall 103. The balloon 152 may have any suitable size, shape, and geometry and may be manufactured using any suitable materials having any suitable properties, such as flexibility, biocompatibility, etc. In addition, the balloon 152 may include one or more surface features, such as protrusions, grooves, coatings, etc. Similar to the assembly 110 described above, the assembly 150 as shown in FIG. 6A may be configured to extend from and withdrawn into a lumen of the elongate member 108. The balloon 152 of the assembly 150 shown in FIG. 6A may be disposed in an expandable basket having a plurality of electrodes defined by legs 111. The legs 111 shown in FIG. 6A may be similar to the legs 111 shown in FIG. 5A and described above. Each of the legs 111 of FIG. 6A may be disposed on an outer diameter of the balloon 152. One of the lumens of the elongate member 108 may include an actuation mechanism, for example, a pull rod 115 connected to the distal tip of the expandable assembly 150. The pull rod 115 of FIG. 6A may be similar to the pull rod 115 described above. The pull rod 115 of FIG. 6A may include a lumen and openings 109 through which the cooling fluid 112 may be delivered to the inner surfaces of the balloon 152 so as to cool the balloon 152.

FIG. 6B is a schematic view of another embodiment a device which may be deployed in the airway 101 of a patient's lung. The expandable energy delivery assembly 150 of FIG. 6B may include an expandable balloon 152 and an elongate member 108. The balloon 152 of FIG. 6B may be similar to the balloon 152 described above in reference to FIG. 6A. Similar to the assembly 110 described above, the assembly 150 as shown in FIG. 6B may be configured to extend from and withdrawn into a lumen of the elongate member 108. The cooling fluid may be delivered within the balloon 152 of FIG. 6B via lumen of the elongate member 108, similar to the manner described above in reference to FIG. 2. The assembly 150 of FIG. 6B also may include a plurality of electrodes defined by legs 111. The legs 111 of FIG. 6B may be similar to the legs 111 shown in FIG. 2A and described above. Each of the legs 111 of FIG. 6B may be disposed adjacent to the expandable balloon 152 of FIG. 6B.

In one aspect of the disclosure, a method of treating an airway 101 of a patient may include delivering thermal energy to an airway wall 103 through a device 100. The device 100 may include an expandable energy delivery assembly 110, 114 that may include a plurality of electrodes configured to deliver thermal energy to the airway wall 103. The method may also include delivering a cooling medium (such as cooling fluid 112) to the airway wall 103 to control a temperature of the airway wall 103.

The cooling medium may be delivered before, during, or after a treatment procedure. Temperature sensors 113 may be employed to measure the temperature of the airway wall 103 during the therapy. In another aspect, the cooling medium may be replaced with a fluid capable of performing, e.g., chemical ablation for destroying or otherwise damaging nerve tissue in the airway wall 103.

An illustrative method of delivering thermal energy to the airway wall 103 is disclosed. The device 100 may be advanced to a treatment location within the airway 101 through a working channel of an introduction member 106 such as a bronchoscope or other suitable introduction sheath. Once deployed at the treatment location a cooling fluid 112 may be injected into a channel adjacent an electrode. Alternatively, the cooling fluid 112 may be disposed in the channel prior to deployment of device 100 within the airway 101. The channel may include a channel opening (such as 147, 109) through which the cooling fluid 112 may be released. Upon release, the cooling fluid 112 may undergo an adiabatic phase change such as liquid to gas due to drop in pressure. This may lower the temperature of the gas and hence, the temperature of the airway wall 103 may also be reduced when the cooling medium (e.g., gas) contacts tissues of the airway. Cooling may penetrate deeper tissue in the airway wall 103 as well. Thermal energy may then be delivered by activating the electrodes. The thermal energy may ablate a targeted region of the airway wall 103, and may destroy or otherwise damage tissues (e.g., nerves) of the airway wall 103. Temperature sensors 113 or impedance sensors may simultaneously measure the temperature or impedance of the airway wall 103 and may provide feedback to a user to regulate the temperature and or impedance. The temperature and/or impedance feedback also may be provided to a controller, which may automatically regulate energy delivered to the electrodes of the device 100. The feedback also may be used to regulate delivery of the cooling fluid 112. Further, as noted above, the cooling fluid 112 may be replaced with a fluid for performing chemical ablation. In some embodiments, the temperature sensors 113 may be replaced with or be in addition to impedance sensors. The impedance sensors may detect a change in impedance associated with and indicative of changes in tissue properties during therapy.

Once the procedure is finished at a particular treatment location, the device 100 may be partially collapsed and advanced to another location for another treatment. Alternatively, the device 100 may be moved along a length of the airway 101 with the expandable energy delivery assembly 110, 114 in an expanded configuration, thereby continuously delivery energy to a length of airway wall 103. Once the procedure is complete, the device 100 may be retracted back into the bronchoscope first and then the bronchoscope may be retracted from the patient's body.

It is contemplated that delivering cooling fluid 112 to the airway wall 103 may lead to effective cooling that may allow regulating the temperature of the therapy delivered to the airway wall 103. Cooling may also prevent inadvertent heating of the surrounding tissue. Hence, the ablation therapy may be precisely controlled and targeted by use of the cooling fluid 112.

Embodiments of the present disclosure may be used in many different medical or non-medical environments. In addition, at least certain aspects of the aforementioned embodiments may be combined with other aspects of the embodiments, or removed, without departing from the scope of the disclosure.

Other embodiments of the present disclosure will be apparent to those skilled in the art from consideration of the specification and practice of the embodiments disclosed herein. It is intended that the specification and examples be considered as exemplary only, with a true scope and spirit of the disclosure being indicated by the following claims.

We claim:

1. A device for delivering energy to tissue within a patient, the device comprising:
an elongate member having a proximal end and a distal end;
an expandable energy delivery assembly extending from the distal end of the elongate member, wherein the expandable energy delivery assembly includes a plurality of legs, wherein at least one of the legs includes an electrode and defines a passageway adjacent to the electrode; and
wherein the passageway is configured to receive a fluid.

2. The device of claim 1, further comprising at least one sensor configured to detect temperature or impedance.

3. The device of claim 1, wherein distal ends of the plurality of legs are coupled together to define a basket.

4. The device of claim 1 wherein each of the plurality of legs includes a free distal end.

5. The device of claim 1, wherein the at least one of the legs includes a plurality of passageways adjacent the electrode.

6. The device of claim 1, wherein a wall of the passageway includes an opening through which the fluid can leave the passageway.

7. The device of claim 6, wherein the opening is configured to cause the fluid to undergo an adiabatic phase change.

8. The device of claim 1, wherein the passageway includes a first cross-sectional configuration and the electrode includes a second cross-sectional configuration different from the first cross-sectional configuration.

9. The device of claim 6, wherein the opening includes a plurality of openings disposed along a wall of the passageway.

10. The device of claim 1, wherein the sensor is disposed on the at least one of the legs.

11. The device of claim 1, wherein the fluid is configured to receive thermal energy from tissue within the patient, and wherein the expandable energy delivery assembly is bipolar.

12. The device of claim 2, further comprising a controller configured to adjust the delivery of energy based on information received from the at least one sensor.

13. The device of claim 1, wherein the fluid comprises nitrous oxide.

14. The device of claim 13, wherein the energy is RF energy.

15. A device for delivering energy to tissue within a patient, the device comprising:
an elongate member including a proximal end and a distal end;
a plurality of legs defining an energy delivery assembly, wherein the energy delivery assembly is configured to selectively transition between an expanded configuration and a collapsed configuration, wherein at least one of the plurality of legs includes an electrode and a passageway for receiving a cooling fluid therein adjacent the electrode; and
a temperature sensor.

16. The device of clam 15, wherein the passageway includes two passageways disposed on either side of the electrode.

17. The device of claim 15, wherein a wall of the passageway includes an opening through which the fluid can leave the passageway, wherein the opening is configured to cause the fluid to undergo an adiabatic phase change.

18. The device of claim 15, wherein the energy delivery assembly is bipolar.

19. A method of treating an airway of a patient, the method comprising:
delivering energy to a wall of the airway via a device, the device comprising:
an elongate member including a proximal end and a distal end;
a plurality of legs defining an energy delivery assembly, wherein the energy delivery assembly is configured to selectively transition between an expanded configuration and a collapsed configuration, wherein at least one of the plurality of the legs includes an electrode and a passageway for receiving a cooling fluid therein adjacent the electrode; and
a sensor.

20. The method of claim 19, wherein the energy delivery assembly is bipolar, and wherein the opening is disposed on at least one of the plurality of electrodes.
